(19) United States
(12) Patent Application Publication
(10) Pub. No.: US 2023/0020272 A1
(43) Pub. Date: Jan. 19, 2023

(54) NEW PROCESS FOR THE MANUFACTURE OF PHARMACEUTICAL COMPOSITIONS

(71) Applicant: NANEXA AB, Uppsala (SE)

(72) Inventors: Anders JOHANSSON, Uppsala (SE); Marten ROOTH, Uppsala (SE); Joel HELLRUP, Uppsala (SE)

(21) Appl. No.: 17/782,734

(22) PCT Filed: Dec. 4, 2020
(86) PCT No.: PCT/GB2020/053129

§ 371 Date: Jun. 6, 2022

(30) Foreign Application Priority Data
Dec. 6, 2019 (GB) 1917899.5

(51) Int. Cl. A61K 9/50; A61K 31/706

(52) U.S. Cl. CPC A61K 9/5089; A61K 31/706; A61K 9/501

(57) ABSTRACT

There is provided a process for the preparation of composition in the form of a plurality of particles having a weight-, number-, and/or volume-based mean diameter that is between amount 10 nm and about 700 μm, which particles comprise: (a) solid cores, preferably comprising a biologically active agent; and (b) two or more sequentially applied, discrete layers, each of which comprises at least one separately applied coating material, and which two or more layers together surround, enclose and/or encapsulate said cores, which process comprises the sequential steps of: (1) applying an initial layer of at least one coating material to said solid cores by way of a gas phase deposition technique; (2) discharging the coated particles from the gas phase deposition reactor and subjecting the coated particles to agitation to disaggregate particle aggregates formed during step (1) by way of mechanical sieving technique; (3) reintroducing the disaggregated, coated particles from step (2) into the gas phase deposition reactor and applying a further layer of at least one coating material to the reintroduced particles; and (1) optionally repeating steps (2) and (3) one or more times to increase the total thickness of the at least one coating material that enclose(s) said solid core. The gas phase deposition technique is preferably atomic layer deposition. When the cores comprise biologically active agent, the compositions may provide for the delayed or sustained release of said active agent without a burst effect.

### FIELD OF THE INVENTION

This invention relates to a new process for the manufacture of compositions that are useful in the field of drug delivery.

### PRIOR ART AND BACKGROUND

The listing or discussion of an apparently prior-published document in this specification should not necessarily be taken as an acknowledgement that the document is part of the state of the art or common general knowledge.

In the field of drug delivery, the ability to control the profile of drug release is of critical importance. It is desirable to ensure that active ingredients are released at a desired and predictable rate in vivo following administration, in order to ensure the optimal pharmacokinetic profile.

In the case of sustained release compositions, it is also of critical importance that a drug delivery composition provides a release profile that minimizes any initial rapid release of active ingredient, that is a large concentration of drug in plasma shortly after administration. Such a burst release may be hazardous in the case of drugs that have a narrow therapeutic window.

In the specific case of injectable suspensions, it is also important to ensure that the size of the suspended particles is controlled so that they can be injected through a needle. If large, aggregated particles are present, they will not only block the needle through which the suspension is to be injected, but also will not form a stable suspension within (i.e. they will instead tend to sink to the bottom of) the injection liquid.

There is thus a general need in the art for effective and/or improved drug transport and delivery systems.

Atomic layer deposition (ALD) is a technique that is employed to deposit thin films comprising a variety of materials, including organic, biological, polymeric and, especially, inorganic materials, such as metal oxides, on solid substrates.

The technique is usually performed at low pressures and elevated temperatures. Film coatings are produced by alternating exposure of solid substrates within an ALD reactor chamber to vaporized reactants in the gas phase. Substrates can be silicon wafers, granular materials or small particles (e.g. microparticles or nanoparticles).

The coated substrate is protected from chemical reactions (decomposition) and physical changes by the solid coating. ALD can also potentially be used to control the rate of release of the substrate material within a solvent, which makes it of potential use in the formulation of active pharmaceutical ingredients.

In ALD, a first precursor, which can be metal-containing, is fed into an ALD reactor chamber (in a so called ‘precursor pulse’), and forms an adsorbed atomic or molecular monolayer at the surface of the substrate. Excess first precursor is then purged from the reactor, and then a second precursor, such as water, is pulsed into the reactor. This reacts with the first precursor, resulting in the formation of a monolayer of e.g. metal oxide on the substrate surface. A subsequent purging pulse is followed by a further pulse of the first precursor, and thus the start of a new cycle of the same events (a so called ‘ALD cycle’).

The thickness of the film coating is controlled by inter alia the number of ALD cycles that are conducted.

In a normal ALD process, because only atomic or molecular monolayers are produced during any one cycle, no discernible physical interface is formed between these monolayers, which essentially become a continuum at the surface of the substrate.

In international patent application WO 2014/187995, a process is described in which a number of ALD cycles are performed, which is followed by periodically removing the resultant coated substrates from the reactor and conducting a re-dispersion/agitation step to present new surfaces available for precursor adsorption.

The agitation step is done primarily to solve a problem observed for nano- and microparticles, namely that, during the ALD coating process, aggregation of particles takes place, resulting in ‘pinholes’ being formed by contact points between such particles. The re-dispersion/agitation step was performed by placing the coated substrates in a solvent, (e.g. water or a hydrocarbon) and sonicating, which resulted in deagglomeration, and the breaking up of contact points between individual particles of coated active substance.

The particles were then loaded back into the reactor and the steps of ALD coating of the powder, and deagglomerating the powder were repeated 3 times, to a total of 4 series of cycles. This process has been found to allow for the formation of coated particles that are, to a large extent, free of pinholes (see also, Hellrup et al, Int. J. Pharm., 529, 116 (2017)).

It has been found that the process of carrying out of ‘sets’ of ALD coating cycles followed by intermittent dispersion, as described in WO 2014/187995, results in clear, separate layers of coatings that are defined by clear, visible, physical interfaces between such coating layers. Such interfaces are clearly visible by a technique such as transmission electron microscopy (TEM) as regions of higher electron permeability. As explained below, similar interfaces are not visible when coatings are built up one atomic layer at a time from the surface of a substrate. This is the case even if different precursors are fed into the ALD reactor in consecutive ALD cycles.

We have now found that it is advantageous to deagglomerate aggregated particles into primary particles externally to the reactor by a dry process that involves a combination of a mechanical forcing means and a sieve. This avoids the need for employing an aggressive deagglomeration technique such as sonication, as well as the need to dry particles prior to placing them back into the reactor for further coating. We have found that conducting the deagglomeration steps in this way allows for the presentation of essentially completely pinhole-free coated particles in a form that can be readily processed into a pharmaceutical formulation.

### DISCLOSURE OF THE INVENTION

According to a first aspect of the invention there is provided a process for the preparation of composition in the form of a plurality of particles of a weight-, number-, and/or volume-based mean diameter that is between amount 10 nm and about 700 μm, which particles comprise (i.e. are made up of):
(a) solid cores, preferably comprising a biologically active agent; and
(b) two or more sequentially applied, discrete layers, each of which comprises at least one separate (i.e. separately applied) coating material, and which two or more layers together surround, enclose and/or encapsulate said cores,

which process comprises the sequential steps of:
(1) applying an initial layer of at least one coating material to said solid cores by way of a gas phase deposition technique;
(2) discharging the coated particles from the gas phase deposition reactor and subjecting the coated particles to agitation to disaggregate particle aggregates formed during step (1) by way of mechanical sieving technique;
(3) reintroducing the disaggregated, coated particles from step (2) into the gas phase deposition reactor and applying a further layer of at least one coating material to the reintroduced particles; and
(4) optionally repeating steps (2) and (3) one or more times to increase the total thickness of the at least one coating material that enclose(s) said solid core,

which process is hereinafter referred to as ‘the process of the invention’.

The term ‘solid’ will be well understood by those skilled in the art to include any form of matter that retains its shape and density when not confined, and/or in which molecules are generally compressed as tightly as the repulsive forces among them will allow. The solid cores have at least a solid exterior surface onto which a layer of coating material can be deposited. The interior of the solid cores may be also solid or may instead be hollow.

For example, if the particles are spray dried before they are placed into the reactor vessel, they may be hollow due to the spray drying technique.

The process of the invention is preferably employed to make pharmaceutical compositions, in which case the composition may comprise a pharmacologically-effective amount of a biologically active agent. Furthermore, said solid cores preferably comprise said biologically active agent.

In this respect, the solid cores may consist essentially of, or comprise, biologically active agent (which agent may hereinafter be referred to interchangeably as a ‘drug’, and ‘active pharmaceutical ingredient (API)’ and/or an ‘active ingredient’). Biologically active agents also include biopharmaceuticals and/or biologics. Biologically active agents can also include a mixture of different APIs, as different API particles or particles comprising more than one API.

By ‘consists essentially’ of biologically-active agent, we include that the solid core is essentially comprised only of biologically active agent(s), i.e. it is free from non-biologically active substances, such as excipients, carriers and the like (vide infra). This means that the core may comprise less than about 5%, such as less than about 3%, including less than about 2%, e.g. less than about 1% of such other excipients.

In the alternative, cores comprising biologically active agents may include such an agent in admixture with one or more pharmaceutical ingredients, which may include pharmaceutically-acceptable excipients, such as adjuvants, diluents or carriers, and/or may include other biologically active ingredients.

Biologically active agents may be presented in a crystalline, a part-crystalline and/or an amorphous state. Biologically active agents may further comprise any substance that is in the solid state, or which may be converted into the solid state, at about room temperature (e.g. about 18° C.) and about atmospheric pressure, irrespective of the physical form. Such agents should also remain in the form of a solid whilst being coated in the reactor and also should not decompose physically or chemically to an appreciable degree (i.e. no more than about 10% w/w) whilst being coated, or after having been covered by at least one of the aforementioned layers of coating material. Biologically active agents may further be presented in combination (e.g. in admixture or as a complex) with another active substance.

As used herein, the term ‘biologically active agent’, or similar and/or related expressions, generally refer(s) to any agent, or drug, capable of producing some sort of physiological effect (whether in a therapeutic or prophylactic capacity against a particular disease state or condition) in a living subject, including, in particular, mammalian and especially human subjects (patients).

Biologically active agents may, for example, be selected from an analgesic, an anaesthetic, an anti-ADHD agent, an anorectic agent, an antiaddictive agent, an antibacterial agent, an antimicrobial agent, an antifungal agent, an antiviral agent, an antiparasitic agent, an antiprotozoal agent, an anthelmintic, an ectoparasiticide, a vaccine, an anticancer agent, an antimetabolite, an alkylating agent, an antineoplastic agent, a topoisomerase, an immunomodulator, an immunostimulant, an immunosuppressant, an anabolic steroid, an anticoagulant agent, an antiplatelet agent, an anticonvulsant agent, an antidementia agent, an antidepressant agent, an antidote, an antihyperlipidemic agent, an antigout agent, an antimalarial, an antimigraine agent, an anti-inflammatory agent, an antiparkinson agent, an antipruritic agent, an antipsoriatic agent, an antiemetic, an anti-obesity agent, an anthelmintic, an antiasthma agent, an antibiotic, an antidiabetic agent, an antiepileptic, an antifibrinolytic agent, an antihemorrhagic agent, an antihistamine, an antitussive, an antihypertensive agent, an antimuscarinic agent, an antimycobacterial agent, an antioxidant agent, an antipsychotic agent, an antipyretic, an antirheumatic agent, an antiarrhythmic agent, an anxiolytic agent, an aphrodisiac, a cardiac glycoside, a cardiac stimulant, an entheogen, an entactogen, an euphoriant, an orexigenic, an antithyroid agent, an anxiolytic sedative, a hypnotic, a neuroleptic, an astringent, a bacteriostatic agent, a beta blocker, a calcium channel blocker, an ACE inhibitor, am angiotensin II receptor antagonist, a renin inhibitor, a beta-adrenoceptor blocking agent, a blood product, a blood substitute, a bronchodilator, a cardiac inotropic agent, a chemotherapeutic, a coagulant, a corticosteroid, a cough suppressant, a diuretic, a deliriant, an expectorant, a fertility agent, a sex hormone, a mood stabilizer, a mucolytic, a neuroprotective, a nootropic, a neurotoxin, a dopaminergic, an antiparkinsonian agent, a free radical scavenging agent, a growth factor, a fibrate, a bile acid sequestrants, a cicatrizant, a glucocorticoid, a mineralcorticoid, a haemostatic, a hallucinogen, a hypothalamic-pituitary hormone, an immunological agent, a laxative agent, a antidiarrhoeals agent, a lipid regulating agent, a muscle relaxant, a parasympathomimetic, a parathyroid calcitonin, a serenic, a statin, a stimulant, a wakefulness-promoting agent, a decongestant, a dietary mineral, a biphosphonate, a cough medicine, an ophthamological, an ontological, a H1 antagonist, a H2 antagonist, a proton pump inhibitor, a prostaglandin, a radio-pharmaceutical, a hormone, a sedative, an anti-allergic agent, an appetite stimulant, a steroid, a sympathomimetic, a thrombolytic, a thyroid agent, a vasodilator, a xanthine, an erectile dysfunction improvement agent, a gastrointestinal agent, a histamine receptor antagonist, a keratolytic, an antianginal agent, a non-steroidal antiinflammatory agent, a COX-2 inhibitor, a leukotriene inhibitor, a macrolide, a NSAID, a nutritional agent, an opioid analgesic, an opioid antagonist, a potassium channel activator, a protease inhibitor, an antiosteoporosis agent, a cognition enhancer, an antiurinary incontinence agent, a nutritional oil, an antibenign prostate hypertrophy agent, an essential fatty acid, a non-essential fatty acid, a radiopharmaceutical, a senotherapeutic, a vitamin, or a mixture of any of these.

The biologically-active agent may also be a cytokine, a peptidomimetic, a peptide, a protein, a toxoid, a serum, an antibody, a vaccine, a nucleoside, a nucleotide, a portion of genetic material, a nucleic acid, or a mixture thereof. Non-limiting examples of therapeutic peptides/proteins are as follows: lepirudin, cetuximab, dornase alfa, denileukin diftitox, etanercept, bivalirudin, leuprolide, alteplase, interferon alfa-n1, darbepoetin alfa, reteplase, epoetin alfa, salmon calcitonin, interferon alfa-n3, pegfilgrastim, sargramostim, secretin, peginterferon alfa-2b, asparaginase, thyrotropin alfa, antihemophilic factor, anakinra, gramicidin D, intravenous immunoglobulin, anistreplase, insulin (regular), tenecteplase, menotropins, interferon gamma-1b, interferon alfa-2a (recombinant), coagulation factor VIIa, oprelvekin, palifermin, glucagon (recombinant), aldesleukin, botulinum toxin Type B, omalizumab, lutropin alfa, insulin lispro, insulin glargine, collagenase, rasburicase, adalimumab, imiglucerase, abciximab, alpha-1-proteinase inhibitor, pegaspargase, interferon beta-1a, pegademase bovine, human serum albumin, eptifibatide, serum albumin iodinated, infliximab, follitropin beta, vasopressin, interferon beta-1b, hyaluronidase, rituximab, basiliximab, muromonab, digoxin immune Fab (ovine), ibritumomab, daptomycin, tositumomab, pegvisomant, botulinum toxin type A, pancrelipase, streptokinase, alemtuzumab, alglucerase, capromab, laronidase, urofollitropin, efalizumab, serum albumin, choriogonadotropin alfa, antithymocyte globulin, filgrastim, coagulation factor IX, becaplermin, agalsidase beta, interferon alfa-2b, oxytocin, enfuvirtide, palivizumab, daclizumab, bevacizumab, arcitumomab, eculizumab, panitumumab, ranibizumab, idursulfase, alglucosidase alfa, exenatide, mecasermin, pramlintide, galsulfase, abatacept, cosyntropin, corticotropin, insulin aspart, insulin detemir, insulin glulisine, pegaptanib, nesiritide, thymalfasin, defibrotide, natural alpha interferon/multiferon, glatiramer acetate, preotact, teicoplanin, canakinumab, ipilimumab, sulodexide, tocilizumab, teriparatide, pertuzumab, rilonacept, denosumab, liraglutide, golimumab, belatacept, buserelin, velaglucerase alfa, tesamorelin, brentuximab vedotin, taliglucerase alfa, belimumab, aflibercept, asparaginase Erwinia chrysanthemi, ocriplasmin, glucarpidase, teduglutide, raxibacumab, certolizumab pegol, insulin isophane, epoetin zeta, obinutuzumab, fibrinolysin aka plasmin, follitropin alpha, romiplostim, lucinactant, natalizumab, aliskiren, ragweed pollen extract, secukinumab, somatotropin (recombinant), drotrecogin alfa, alefacept, OspA lipoprotein, urokinase, abarelix, sermorelin, aprotinin, gemtuzumab ozogamicin, satumomab pendetide, albiglutide, antithrombin alfa, antithrombin III (human), asfotase alfa, atezolizumab, autologous cultured chondrocytes, beractant, blinatumomab, Cl esterase inhibitor (human), coagulation factor XIII A-subunit (recombinant), conestat alfa, daratumumab, desirudin, dulaglutide, elosulfase alfa, evolocumab, fibrinogen concentrate (human), filgrastim-sndz, gastric intrinsic factor, hepatitis B immune globulin, human calcitonin, human Clostridium tetani toxoid immune globulin, human rabies virus immune globulin, human Rho(D) immune globulin, human Rho(D) immune globulin, hyaluronidase (human, recombinant), idarucizumab, immune globulin (human), vedolizumab, ustekinumab, turoctocog alfa, tuberculin purified protein derivative, simoctocog alfa, siltuximab, sebelipase alfa, sacrosidase, ramucirumab, prothrombin complex concentrate, poractant alfa, pembrolizumab, peginterferon beta-1a, ofatumumab, obiltoxaximab, nivolumab, necitumumab, metreleptin, methoxy polyethylene glycol-epoetin beta, mepolizumab, ixekizumab, insulin degludec, insulin (porcine), insulin (bovine), thyroglobulin, anthrax immune globulin (human), anti-inhibitor coagulant complex, brodalumab, Cl esterase inhibitor (recombinant), chorionic gonadotropin (human), chorionic gonadotropin (recombinant), coagulation factor X (human), dinutuximab, efmoroctocog alfa, factor IX complex (human), hepatitis A vaccine, human varicella-zoster immune globulin, ibritumomab tiuxetan, lenograstim, pegloticase, protamine sulfate, protein S (human), sipuleucel-T, somatropin (recombinant), susoctocog alfa and thrombomodulin alfa.

Non-limiting examples of drugs which may be used according to the present invention are all-trans retinoic acid (tretinoin), alprazolam, allopurinol, amiodarone, amlodipine, asparaginase, astemizole, atenolol, azathioprine, azelatine, beclomethasone, bendamustine, bleomycin, budesonide, buprenorphine, butalbital, capecitabine, carbamazepine, carbidopa, carboplatin, cefotaxime, cephalexin, chlorambucil, cholestyramine, ciprofloxacin, cisapride, cisplatin, clarithromycin, clonazepam, clozapine, cyclophosphamide, cyclosporin, cytarabine, dacarbazine, dactinomycin, daunorubicin, diazepam, diclofenac sodium, digoxin, dipyridamole, divalproex, dobutamine, docetaxel, doxorubicin, doxazosin, enalapril, epirubicin, erlotinib, estradiol, etodolac, etoposide, everolimus, famotidine, felodipine, fentanyl citrate, fexofenadine, filgrastim, finasteride, fluconazole, flunisolide, fluorouracil, flurbiprofen, fluralaner, fluvoxamine, furosemide, gemcitabine, glipizide, gliburide, ibuprofen, ifosfamide, imatinib, indomethacin, irinotecan, isosorbide dinitrate, isotretinoin, isradipine, itraconazole, ketoconazole, ketoprofen, lamotrigine, lansoprazole, loperamide, loratadine, lorazepam, lovastatin, medroxyprogesterone, mefenamic acid, mercaptopurine, mesna, methotrexate, methylprednisolone, midazolam, mitomycin, mitoxantrone, moxidectine, mometasone, nabumetone, naproxen, nicergoline, nifedipine, norfloxacin, omeprazole, oxaliplatin, paclitaxel, phenyloin, piroxicam, procarbazine, quinapril, ramipril, risperidone, rituximab, sertraline, simvastatin, sulindac, sunitinib, temsirolimus, terbinafine, terfenadine, thioguanine, trastuzumab, triamcinolone, valproic acid, vinblastine, vincristine, vinorelbine, zolpidem, or pharmaceutically acceptable salts of any of these.

Compositions made by the process of the invention may comprise benzodiazipines, such as alprazolam, chlordiazepoxide, clobazam, clorazepate, diazepam, estazolam, flurazepam, lorazepam, oxazepam, quazepam, temazepam, triazolam and pharmaceutically acceptable salts of any of these.

Anaesthetics that may also be employed in the compositions made by the process of the invention may be local or general. Local anaesthetics that may be mentioned include amylocaine, ambucaine, articaine, benzocaine, benzonatate, bupivacaine, butacaine, butanilicaine, chloroprocaine, cinchocaine, cocaine, cyclomethycaine, dibucaine, diperodon, dimethocaine, eucaine, etidocaine, hexylcaine, fomocaine, fotocaine, hydroxyprocaine, isobucaine, levobupivacaine, lidocaine, mepivacaine, meprylcaine, metabutoxycaine, nitracaine, orthocaine, oxetacaine, oxybuprocaine, paraethoxycaine, phenacaine, piperocaine, piridocaine, pramocaine, prilocaine, primacaine, procaine, procainamide, proparacaine, propoxycaine, pyrrocaine, quinisocaine, ropivacaine, trimecaine, tolycaine, tropacocaine, or pharmaceutically acceptable salts of any of these.

Psychiatric drugs may also be employed in the compositions made by the process of the invention. Psychiatric drugs that may be mentioned include 5-HTP, acamprosate, agomelatine, alimemazine, amfetamine, dexamfetamine, amisulpride, amitriptyline, amobarbital, amobarbital/secobarbital, amoxapine, amphetamine(s), aripiprazole, asenapine, atomoxetine, baclofen, benperidol, bromperidol, bupropion, buspirone, butobarbital, carbamazepine, chloral hydrate, chlorpromazine, chlorprothixene, citalopram, clomethiazole, clomipramine, clonidine, clozapine, cyclobarbital/diazepam, cyproheptadine, cytisine, desipramine, desvenlafaxine, dexamfetamine, dexmethylphenidate, diphenhydramine, disulfiram, divalproex sodium, doxepin, doxylamine, duloxetine, enanthate, escitalopram, eszopiclone, fluoxetine, flupenthixol, fluphenazine, fluspirilen, fluvoxamine, gabapentin, glutethimide, guanfacine, haloperidol, hydroxyzine, iloperidone, imipramine, lamotrigine, levetiracetam, levomepromazine, levomilnacipran, lisdexamfetamine, lithium salts, lurasidone, melatonin, melperone, meprobamate, metamfetamine, nethadone, methylphenidate, mianserin, mirtazapine, moclobemide, nalmefene, naltrexone, niaprazine, nortriptyline, olanzapine, ondansetron, oxcarbazepine, paliperidone, paroxetine, penfluridol, pentobarbital, perazine, pericyazine, perphenazine, phenelzine, phenobarbital, pimozide, pregabalin, promethazine, prothipendyl, protriptyline, quetiapine, ramelteon, reboxetine, reserpine, risperidone, rubidium chloride, secobarbital, selegiline, sertindole, sertraline, sodium oxybate, sodium valproate, sodium valproate, sulpiride, thioridazine, thiothixene, tianeptine, tizanidine, topiramate, tranylcypromine, trazodone, trifluoperazine, trimipramine, tryptophan, valerian, valproic acid in 2.3:1 ratio, varenicline, venlafaxine, vilazodone, vortioxetine, zaleplon, ziprasidone, zolpidem, zopiclone, zotepine, zuclopenthixol and pharmaceutically acceptable salts of any of these.

Opioid analgesics that may be employed in compositions made by the process of the invention include buprenorphine, butorphanol, codeine, fentanyl, hydrocodone, hydromorphone, meperidine, methadone, morphine, nomethadone, opium, oxycodone, oxymorphone, pentazocine, tapentadol, tramadol and pharmaceutically acceptable salts of any of these.

Opioid antagonists that may be employed in compositions made by the process of the invention include naloxone, nalorphine, niconalorphine, diprenorphine, levallorphan, samidorphan, nalodeine, alvimopan, methylnaltrexone, naloxegol, 6β-naltrexol, axelopran, bevenopran, methylsamidorphan, naldemedine, preferably nalmefene and, especially, naltrexone, as well as pharmaceutically acceptable salts of any of these.

Anticancer agents that may be included in compositions made by the process of the invention include the following: actinomycin, afatinib, all-trans retinoic acid, amsakrin, anagrelid, arseniktrioxid, axitinib, azacitidine, azathioprine, bendamustine, bexaroten, bleomycin, bortezomib, bosutinib, busulfan, cabazitaxel, capecitabine, carboplatin, chlorambucil, cladribine, clofarabine, cytarabine, dabrafenib, dacarbazine, dactinomycin, dasatinib, daunorubicin, decitabine, docetaxel, doxifluridine, doxorubicin, epirubicin, epothilone, erlotinib, estramustin, etoposide, everolimus, fludarabine, fluorouracil, gefitinib, guadecitabine, gemcitabine, hydroxycarbamide, hydroxyurea, idarubicin, idelalisib, ifosfamide, imatinib, irinotecan, ixazomib, kabozantinib, karfilzomib, krizotinib, lapatinib, lomustin, mechlorethamine, melphalan, mercaptopurine, mesna, methotrexate, mitotan, mitoxantrone, nelarabin, nilotinib, niraparib, olaparib, oxaliplatin, paclitaxel, panobinostat, pazopanib, pemetrexed, pixantron, ponatinib, procarbazine, regorafenib, ruxolitinib, sonidegib, sorafenib, sunitinib, tegafur, temozolomid, teniposide, tioguanine, tiotepa, topotecan, trabektedin, valrubicin, vandetanib, vemurafenib, venetoklax, vinblastine, vincristine, vindesine, vinflunin, vinorelbine, vismodegib, as well as pharmaceutically acceptable salts of any of these. A preferred biologically active agent is azacitidine.

Such compounds may be used in any one of the following cancers: adenoid cystic carcinoma, adrenal gland cancer, amyloidosis, anal cancer, ataxia-telangiectasia, atypical mole syndrome, basal cell carcinoma, bile duct cancer, Birt-Hogg Dube, tube syndrome, bladder cancer, bone cancer, brain tumor, breast cancer (including breast cancer in men), carcinoid tumor, cervical cancer, colorectal cancer, ductal carcinoma, endometrial cancer, esophageal cancer, gastric cancer, gastrointestinal stromal tumor, HER2-positive, breast cancer, islet cell tumor, juvenile polyposis syndrome, kidney cancer, laryngeal cancer, acute lymphoblastic leukemia, all types of acute lymphocytic leukemia, acute myeloid leukemia, adult leukemia, childhood leukemia, chronic lymphocytic leukemia, chronic myeloid leukemia, liver cancer, lobular carcinoma, lung cancer, small cell lung cancer, Hodgkin's lymphoma, non-Hodgkin's lymphoma, malignant glioma, melanoma, meningioma, multiple myeloma, myelodysplastic syndrome, nasopharyngeal cancer, neuroendocrine tumor, oral cancer, osteosarcoma, ovarian cancer, pancreatic cancer, pancreatic neuroendocrine tumors, parathyroid cancer, penile cancer, peritoneal cancer, Peutz-Jeghers syndrome, pituitary gland tumor, polycythemia vera, prostate cancer, renal cell carcinoma, retinoblastoma, salivary gland cancer, sarcoma, Kaposi sarcoma, skin cancer, small intestine cancer, stomach cancer, testicular cancer, thymoma, thyroid cancer, uterine (endometrial) cancer, vaginal cancer, Wilms' tumor.

Cancers that may be mentioned include myelodysplastic syndrome and sub-types, such as acute myeloid leukemia, refractory anemia or refractory anemia with ringed sideroblasts (if accompanied by neutropenia or thrombocytopenia or requiring transfusions), refractory anemia with excess blasts, refractory anemia with excess blasts in transformation, and chronic myeloid (myelomonocytic) leukemia leukemia.

Other drugs that may be mentioned for use in compositions made by the process of the invention include immunomodulatory imide drugs, such as thalidomide and analogues thereof, such as pomalidomide, lenalidomide and apremilast, and pharmaceutically acceptable salts of any of these. Other drugs that many be mentioned include angiotensin II receptor type 2 agonists, such as Compound 21 (C21; 3-[4-(1H-imidazol-1-ylmethyl)phenyl]-5-(2-methylpropyl)thiophene-2-[(N-butyloxylcarbamate)sulphonamide] and pharmaceutically acceptable (e.g. sodium) salts thereof.

Compositions made by the process of the invention may comprise a pharmacologically-effective amount of biologically-active agents. The term ‘pharmacologically-effective amount’ refers to an amount of such active ingredient, which is capable of conferring a desired physiological change (such as a therapeutic effect) on a treated patient, whether administered alone or in combination with another active ingredient. Such a biological or medicinal response, or such an effect, in a patient may be objective (i.e. measurable by some test or marker) or subjective (i.e. the subject gives an indication of, or feels, an effect), and includes at least partial alleviation of the symptoms of the disease or disorder being treated, or curing or preventing said disease or disorder.

Doses of active ingredients that may be administered to a patient should thus be sufficient to effect a therapeutic response over a reasonable and/or relevant timeframe. One skilled in the art will recognize that the selection of the exact dose and composition and the most appropriate delivery regimen will also be influenced by not only the nature of the active ingredient, but also inter alia the pharmacological properties of the formulation, the route of administration, the nature and severity of the condition being treated, and the physical condition and mental acuity of the recipient, as well as the age, condition, body weight, sex and response of the patient to be treated, and the stage/severity of the disease, as well as genetic differences between patients.

Administration of compositions made by the process of the invention may be continuous or intermittent (e.g. by bolus injection). Dosages of active ingredients may also be determined by the timing and frequency of administration.

In any event, the medical practitioner, or other skilled person, will be able to determine routinely the actual dosage of any particular active ingredient, which will be most suitable for an individual patient.

Alternatively, compositions as described herein may also comprise, instead of (or in addition to) biologically-active agents, diagnostic agents (i.e. agents with no direct therapeutic activity per se, but which may be used in the diagnosis of a condition, such as a contrast agents or contrast media for bioimaging).

Non-biologically active adjuvants, diluents and carriers that may be employed in cores to be coated in accordance with the invention may include pharmaceutically-acceptable substances that are soluble in water, such as carbohydrates, e.g. sugars, such as lactose and/or trehalose, and sugar alcohols, such as mannitol, sorbitol and xylitol; or pharmaceutically-acceptable inorganic salts, such as sodium chloride. Preferred carrier/excipient materials include sugars and sugar alcohols. Such carrier/excipient materials are particularly useful when the biologically active agent is a complex macromolecule, such as a peptide, a protein or portions of genetic material or the like, for example as described generally and/or the specific peptides/proteins described hereinbefore including vaccines. Embedding complex macromolecules in excipients in this way will often result in larger cores for coating, and therefore larger coated particles.

It is not a requirement that the cores of the compositions made by the process of the invention comprise a biologically active agent. Whether the cores do or do not comprise a biologically active agent, the cores may comprise and/or consist essentially of one or more non-biologically active adjuvants, diluents and carriers, including emollients, and/or other excipients with a functional property, such as a buffering agent and/or a pH modifying agent (e.g. citric acid).

The cores are provided in the form of nanoparticles or, more preferably, microparticles. Preferred weight-, number-, or volume-, based mean diameters are between about 50 nm (e.g. about 100 nm, such as about 250 nm) and about 30 μm, for example between about 500 nm and about 100 μm, more particularly between about 1 μm and about 50 μm, such as about 25 μm, e.g. about 20 μm.

As used herein, the term ‘weight based mean diameter’ will be understood by the skilled person to include that the average particle size is characterised and defined from a particle size distribution by weight, i.e. a distribution where the existing fraction (relative amount) in each size class is defined as the weight fraction, as obtained by e.g. sieving (e.g. wet sieving). As used herein, the term ‘number based mean diameter’ will be understood by the skilled person to include that the average particle size is characterised and defined from a particle size distribution by number, i.e. a distribution where the existing fraction (relative amount) in each size class is defined as the number fraction, as measured by e.g. microscopy. As used herein, the term ‘volume based mean diameter’ will be understood by the skilled person to include that the average particle size is characterised and defined from a particle size distribution by volume, i.e. a distribution where the existing fraction (relative amount) in each size class is defined as the volume fraction, as measured by e.g. laser diffraction. Other instruments that are well known in the field may be employed to measure particle size, such as equipment sold by e.g. Malvern Instruments, Ltd (Worcestershire, UK) and Shimadzu (Kyoto, Japan).

Particles may be spherical, that is they possess an aspect ratio smaller than about 20, more preferably less than about 10, such as less than about 4, and especially less than about 2, and/or may possess a variation in radii (measured from the centre of gravity to the particle surface) in at least about 90% of the particles that is no more than about 50% of the average value, such as no more than about 30% of that value, for example no more than about 20% of that value.

Nevertheless, the coating of particles on any shape is also possible in accordance with the invention. For example, irregular shaped (e.g. ‘raisin’-shaped), needle-shaped, or cuboid-shaped particles may be coated. For a non-spherical particle, the size may be indicated as the size of a corresponding spherical particle of e.g. the same weight, volume or surface area. Hollow particles, as well as particles having pores, crevices etc., such as fibrous or ‘tangled’ particles may also be coated in accordance with the invention.

Particles may be obtained in a form in which they are suitable to be coated or be obtained in that form, for example by particle size reduction processes (e.g. crushing, cutting, milling or grinding to a specified weight based mean diameter (as hereinbefore defined), for example by wet grinding, dry grinding, air jet milling (including cryogenic micronization), ball milling, such as planetary ball milling, as well as making use of end-runner mills, roller mills, vibration mills, hammer mills, roller mill, fluid energy mills, pin mills, etc. Alternatively, particles may be prepared directly to a suitable size and shape, for example by spray-drying, precipitation, including the use of supercritical fluids or other top-down methods (i.e. reducing the size of large particles, by e.g. grinding, etc.), or bottom-up methods (i.e. increasing the size of small particles, by e.g. sol-gel techniques, etc.). Nanoparticles may alternatively be made by well-known techniques, such as gas condensation, attrition, chemical precipitation, ion implantation, pyrolysis, hydrothermal synthesis, etc.

It may be necessary (depending upon how the particles that comprise the cores are initially provided) to wash and/or clean them to remove impurities that may derive from their production, and then dry them. Drying may be carried out by way of numerous techniques known to those skilled in the art, including evaporation, spray-drying, vacuum drying, freeze drying, fluidized bed drying, microwave drying, IR radiation, drum drying, etc. If dried, cores may then be deagglomerated by grinding, screening, milling and/or dry sonication. Alternatively, cores may be treated to remove any volatile materials that may be absorbed onto its surface, e.g. by exposing the particle to vacuum and/or elevated temperature.

Surfaces of cores may be chemically activated prior to applying the first layer of coating material, e.g. by treatment with hydrogen peroxide, ozone, free radical-containing reactants or by applying a plasma treatment, in order to create free oxygen radicals at the surface of the core. This in turn may produce favourable adsorption/nucleation sites on the cores for the ALD precursors.

More than one layer of coating material is applied to the core sequentially. Preferred gas phase deposition techniques include ALD or related technologies, such as atomic layer epitaxy (ALE), molecular layer deposition (MLD; a similar technique to ALD with the difference that molecules (commonly organic molecules) are deposited in each pulse instead of atoms), molecular layer epitaxy (MLE), chemical vapor deposition (CVD), atomic layer CVD, molecular layer CVD, physical vapor deposition (PVD), sputtering PVD, reactive sputtering PVD, evaporation PVD and binary reaction sequence chemistry. ALD is the preferred method of coating according to the invention.

Two or more separate layers or coating material (also referred to herein as ‘coatings’ or ‘shells’, all of which terms are used herein interchangeably) are applied (that is ‘separately applied’) to the solid cores comprising biologically active agent. Such ‘separate application’ of ‘separate layers, coatings or shells’ means that the solid cores are coated with a first layer of coating material, and then that resultant coated core is subjected to some form of mechanical sieving technique, step or process. In this respect, the number of discrete layers of coating material(s) as defined herein corresponds to the number of these intermittent mechanical sieving steps, with a final mechanical sieving step being conducted prior to the application of a final layer of coating material.

The mechanical sieving technique that is an essential part of the process will involve a means of mechanically forcing the solid product mass formed by coating said cores through a sieve that is located externally to (i.e. outside of) the reactor, and is configured to deagglomerate any particle aggregates upon said mechanical forcing of the coated cores, prior to being subjected to a second and/or a further layer of coating material. This process is repeated as many times as is required and/or appropriate prior to the application of a final layer of coating material.

Mechanical forcing means may thus comprise one or more of any means of forcing the coated mass through a sieve in a mechanical and/or automated way, in a manner in which that forcing means is not applied manually by way of human force. Mechanical forces may thus take the form of tapping, oscillation, application of a pressure gradient (e.g. a jet), horizontal rotation, mechanised periodical displacement of a sieve, centrifugal forces, sieving or combinations thereof, such as oscillating and tapping, rotating and tapping, etc.

We prefer however that the mechanical forcing means is vibrational. Here, an appropriate means of applying vibrational force (i.e. shaking) forces the coated mass of powder through a mesh or sieve. Said vibration or shaking may be provided by any mechanical means of generating oscillations about an equilibrium point, which generation means may be via acoustic waves (including sonic and ultrasonic waves), or may be mechanical (e.g. tapping), or other ways, including combinations thereof, such as ultrasonic and sonic, sonic and tapping, ultrasonic and tapping, etc.

Appropriate sieve meshes may include perforated plates, microplates, grid, diamond, but are preferably made from threads or wires (woven wire sieves).

We prefer that at least one of the mechanical sieving steps in a process of the invention is carried out by way of a sonic sifter, as described hereinafter. Manufacturers of suitable sonic sifters include Advantech Manufacturing, Endecott and Tsutsui.

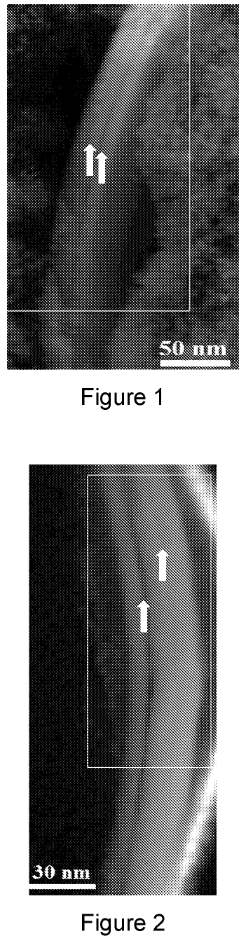

We have found that applying separate layers of coating materials following external deagglomeration gives rise to visible and discernible interfaces that may be observed by analysing coated particles according to the invention, and are observed by e.g. TEM as regions of higher electron permeability, for example, as can be seen in FIGS. 1 and 2.

This is to be contrasted to continuous ALD processes in which coated particles are not removed from the reactor prior to re-coating. Because, in an ALD coating process, coating takes place at the atomic level, even if different coating materials are sequentially employed (e.g. switching from one metal oxide precursor to another between ALD cycles), clear, physical interfaces, such as those shown in FIGS. 1 and 2 are not observed. Thus, the thickness of the layers between the interfaces that can be seen in FIGS. 1 and 2 correspond directly to the number of cycles in each series that are carried out within the ALD reactor, and between individual external agitation steps.

Without being limited by theory, it is believed that removing coated particles from the vacuum conditions of the ALD reactor and exposing a newly-coated surface to the atmosphere results in structural rearrangements due to relaxation and reconstruction of the outermost atomic layers. Such a process is believed to involve rearrangement of surface (and near surface) atoms, driven by a thermodynamic tendency to reduce surface free energy.

Furthermore, surface adsorption of species, e.g. hydrocarbons that are always present in the air, may contribute to this phenomenon, as can surface modifications, due to reaction of coatings formed with hydrocarbons, as well as atmospheric oxygen and the like. Accordingly, if such interfaces are analysed chemically, they may contain traces of contaminants that do not originate from the coating process, such as ALD.

Particle aggregates are thus broken up by a mechanical forcing means that forces them through a sieve, separating the aggregates into individual particles or aggregates of a desired and predetermined size (and thereby achieving deagglomeration). In the latter regard, in some cases the individual primary particle size is so small (i.e. <1 μm) that achieving ‘full’ deagglomeration (i.e. where aggregates are broken down into individual particles) is not possible. Instead, deagglomeration is achieved by breaking down larger aggregates into smaller aggregates of secondary particles of a desired size, as dictated by the size of the sieve mesh. The smaller aggregates are then coated by the gas phase technique to form fully coated ‘particles’ in the form of small aggregate particles. In this way, the term ‘particles’, when referring the particles that have been deagglomerated and coated in the context of the invention, refers to both individual (primary) particles and aggregate (secondary) particles of a desired size.

In any event, the desired particle size (whether that be of individual particles or aggregates of a desired size) is maintained and, moreover, continued application of the gas phase coating mechanism to the particles after such deagglomeration via the mechanical sieving means that a complete coating is formed on the particle, thus forming fully-coated particles (individual or aggregates of a desired size).

The process of the invention may be carried out in a manner that involves carrying out steps (2) and (3) of that process at least 1, preferably 2, more preferably 3, such as 4, including 5, more particularly 6, e.g. 7 times, and no more than about 100 times, for example no more than about 50 times, such as no more than about 40 times, including no more than about 30 times, such as between 2 and 20 times, e.g. between 3 and 15 times, such as 10 times, e.g. 9 or 8 times, more preferably 6 or 7 times, and particularly 4 or 5 times.

The total thickness of the coating (meaning all the separate layers/coatings/shells) will on average be in the region of between about 0.5 nm and about 2 μm.

The minimum thickness of each individual layer/coating/shell will on average be in the region of about 0.5 nm (for example about 0.75 nm, such as about 1 nm).

The maximum thickness of each individual layer/coating/shell will depend on the size of the core (to begin with), and thereafter the size of the core with the coatings that have previously been applied, and may be on average about 1 hundredth of the mean diameter (i.e. the weight-, number-, or volume-, based mean diameter) of that core, or core with previously-applied coatings.

Preferably, for particles with a mean diameter that is between about 100 nm and about 1 μm, the coating thickness should be on average between about 1 nm and about 5 nm; for particles with a mean diameter that is between about 1 μm and about 20 μm, the coating thickness should be on average between about 1 nm and about 10 nm; for particles with a mean diameter that is between about 20 μm and about 700 μm, the coating thickness should be on average between about 1 nm and about 100 nm.

We have found that applying coatings/shells followed by conducting one or more deagglomeration step such as sonication gives rise to abrasions, pinholes, breaks, gaps, cracks and/or voids (hereinafter ‘cracks’) in the layers/coatings, due to coated particles essentially being more tightly ‘bonded’ or ‘glued’ together directly after the application of a thicker coating. This may expose a core comprising biologically-active ingredient to the elements once deagglomeration takes place.

As described herein, we have surprisingly found that by conducting a mechanical sifting process in accordance with the invention (as opposed to sonication as described in international patent application WO 2014/187995, or manually forcing the particles through a sieve by hand) gives rise to significantly less pinholes, gaps or cracks in the final layer of coating material, giving rise to particles that are not only completely covered by that layer/coating, but are also covered in a manner that enables the particles to be deagglomerated readily (e.g. using a non-aggressive technique, such as vortexing) in a manner that does not destroy the layers of coating material that have been formed, prior to, and/or during, pharmaceutical formulation.

For example, if it is intended to provide a sample in suspension prior to administration to a patient, it is necessary to provide deagglomerated primary particles without pinholes or cracks in the coatings. Such cracks will result in an undesirable initial peak (burst) in plasma concentration of active ingredient directly after administration.

As described hereinafter, the process of the invention results in the deagglomerated coated particles with the essential absence of said cracks through which active ingredient can be released in an uncontrolled way. By ‘essentially free of said cracks’ in the coating(s), we mean that less than about 1% of the surfaces of the coated particles comprise abrasions, pinholes, breaks, gaps, cracks and/or voids through which active ingredient is potentially exposed (to, for example, the elements).

The layers of coating material may, taken together, be of an essentially uniform thickness over the surface area of the particles. By ‘essentially uniform’ thickness, we mean that the degree of variation in the thickness of the coating of at least about 10%, such as about 25%, e.g. about 50%, of the coated particles that are present in a composition of the invention, as measured by TEM, is no more than about ±20%, including ±50% of the average thickness.

Coating materials that may be applied to cores may be pharmaceutically-acceptable, in that they should be essentially non-toxic.

Coating materials may comprise organic or polymeric materials, such as a polyamide, a polyimide, a polyurea, a polyurethane, a polythiourea, a polyester or a polyimine. Coating materials may also comprise hybrid materials (as between organic and inorganic materials), including materials that are a combination between a metal, or another element, and an alcohol, a carboxylic acid, an amine or a nitrile. However, we prefer that coating materials comprise inorganic materials.

Inorganic coating materials may comprise one or more metals or metalloids, or may comprise one or more metal-containing, or metalloid-containing, compounds, such as metal, or metalloid, oxides, nitrides, sulphides, selenides, carbonates, and/or other ternary compounds, etc. Metal, and metalloid, hydroxides and, especially, oxides are preferred, especially metal oxides.

Metals that may be mentioned include alkali metals, alkaline earth metals, noble metals, transition metals, post-transition metals, lanthanides, etc. Metal and metalloids that may be mentioned include aluminium, titanium, magnesium, iron, gallium, zinc, zirconium, niobium, hafnium, tantalum, lanthanum, and/or silicon; more preferably aluminium, titanium, magnesium, iron, gallium, zinc, zirconium, and/or silicon; especially aluminium, titanium and/or zinc.

As mentioned above, as the compositions made by the process of the invention comprises two or more discrete layers of inorganic coating materials, the nature and chemical composition(s) of those layers may differ from layer to layer.

Individual layers may also comprise a mixture of two or more inorganic materials, such as metal oxides or metalloid oxides, and/or may comprise multiple layers or composites of different inorganic or organic materials, to modify the properties of the layer.

Coating materials that may be mentioned include those comprising aluminium oxide (Al₂O₃), titanium dioxide (TiO₂), iron oxides (FeₓO_{y}, e.g. FeO and/or Fe₂O₃ and/or Fe₃O₄), gallium oxide (Ga₂O₃), magnesium oxide (MgO), zinc oxide (ZnO), niobium oxide (Nb₂O₅), hafnium oxide (HfO₂), tantalum oxide (Ta₂O₅), lanthanum oxide (La₂O₃), zirconium dioxide (ZrO₂) and/or silicon dioxide (SiO₂). Preferred coating materials include aluminium oxide, titanium dioxide, iron oxides, gallium oxide, magnesium oxide, zinc oxide, zirconium dioxide and silicon dioxide. More preferred coating materials include iron oxide, as well as titanium dioxide, zinc sulphide, zinc oxide and aluminium oxide.

Layers of coating materials (on an individual or a collective basis) in compositions made by the process of the invention may consist essentially (e.g. is greater than about 80%, such as greater than about, 90%, e.g. about 95%, such as about 98%) of iron oxides, aluminium oxide, zinc oxide or titanium dioxide.

The process of the invention is particularly useful when the coating material(s) that is/are applied to the cores comprise zinc oxide.

In ALD, layers of coating materials may be applied at process temperatures from about 20° C. to about 800° C., or from about 40° C. to about 200° C., e.g. from about 40° C. to about 150°, such as from about 50° C. to about 100° C. The optimal process temperature depends on the reactivity of the precursors and/or the substances (including biologically-active agents) that are employed in the core and/or melting point of the core substance(s). When the cores to be coated comprise a biologically-active ingredient, it is preferred that a lower temperature, such as from about 30° C. to about 100° C. is employed.

In most instances, the first of the consecutive reactions will involve some functional group or free electron pairs or radicals at the surface to be coated, such as a hydroxy group (—OH) or a primary or secondary amino group (—NH₂ or —NHR where R e.g. is an aliphatic group, such as an alkyl group). The individual reactions are advantageously carried out separately and under conditions such that all excess reagents and reaction products are essentially removed before conducting the subsequent reaction.

Although the plurality of coated particles according to the invention are essentially free of the aforementioned cracks in the applied coatings, through which active ingredient is potentially exposed (to, for example, the elements), a further, optional step may be applied to the plurality of coated particles prior to subjecting it to further pharmaceutical formulation processing. This optional step may comprise ensuring that the few remaining particles with broken and/or cracked shells/coatings are subjected to a treatment in which all particles are suspended in a solvent in which the active ingredient is soluble (e.g. with a solubility of at least about 1 mg/mL), but the least soluble material in the coating is insoluble (e.g. with a solubility of no more than about 0.1 μg/mL), followed by separating solid matter particles from solvent by, for example, centrifugation, sedimentation, flocculation and/or filtration, resulting in mainly intact particles being left.

The above-mentioned optional step provides a means of potentially reducing further the likelihood of a (possibly) undesirable initial peak (burst) in plasma concentration of active ingredient, as discussed herein.

At the end of the process, coated particles may be dried using one or more of the techniques that are described hereinbefore for drying cores. Drying may take place in the absence, or in the presence, of one or more pharmaceutically acceptable excipients (e.g. a sugar or a sugar alcohol).

Alternatively, at the end of the process, separated particles may be resuspended in a solvent (e.g. water, with or without the presence of one or more pharmaceutically acceptable excipients as defined herein), for subsequent storage and/or administration to patients.

Prior to applying the first layer of coating material or between successive coatings, cores and/or partially coated particles may be subjected to one or more alternative and/or preparatory surface treatments. In this respect, one or more intermediary layers comprising different materials (i.e. other than the inorganic material(s)) may be applied to the relevant surface, e.g. to protect the cores or partially-coated particles from unwanted reactions with precursors during the coating step(s)/deposition treatment, to enhance coating efficiency, or to reduce agglomeration.

An intermediary layer may, for example, comprise one or more surfactants, with a view to reducing agglomeration of particles to be coated and to provide a hydrophilic surface suitable for subsequent coatings. Suitable surfactants in this regard include well known non-ionic, anionic, cationic or zwitterionic surfactants, such as the Tween series, e.g. Tween 80. Alternatively, cores may be subjected to a preparatory surface treatment if the active ingredient that is employed as part of (or as) that core is susceptible to reaction with one or more precursor compounds that may be present in the gas phase during the coating (e.g. the ALD) process.

Application of ‘intermediary’ layers/surface treatments of this nature may alternatively be achieved by way of a liquid phase non-coating technique, followed by a lyophilisation, spray drying or other drying method, to provide particles with surface layers to which coating materials may be subsequently applied.

Outer surfaces of particles of compositions made by the process of the invention may also be derivatized or functionalized, e.g. by attachment of one or more chemical compounds or moieties to the outer surfaces of the final layer of coating material, e.g. with a compound or moiety that enhances the targeted delivery of the particles within a patient to whom the nanoparticles are administered. Such a compound may be an organic molecule (such as PEG) polymer, an antibody or antibody fragment, or a receptor-binding protein or peptide, etc.

Alternatively, the moiety may be an anchoring group such as a moiety comprising a silane function (see, for example, Herrera et al, J. Mater. Chem., 18, 3650 (2008) and U.S. Pat. No. 8,097,742). Another compound, e.g. a desired targeting compound may be attached to such an anchoring group by way of covalent bonding, or non-covalent bonding, including bonding, hydrogen bonding, or van der Waals bonding, or a combination thereof.

The presence of such anchoring groups may provide a versatile tool for targeted delivery to specific sites in the body. Alternatively, the use of compound such as PEG may cause particles to circulate for a longer duration in the blood stream, ensuring that they do not become accumulated in the liver or the spleen (the natural mechanism by which the body eliminates particles, which may prevent delivery to diseased tissue).

Compositions made by the process of the invention are either suitable for administration to patients as they are prepared (i.e. as a plurality of particles) or are preferably formulated together with one or more pharmaceutically-acceptable excipients, including adjuvants, diluents or carriers for use in the medicinal or veterinary fields (including in therapy and/or, if the core comprises a diagnostic material, in diagnostics).

There is further provided compositions made by the process of the invention for use in medicine, diagnostics, and/or in veterinary practice and a pharmaceutical (or veterinary) formulation comprising a composition of the invention and a pharmaceutically- (or veterinarily-) acceptable adjuvant, diluent or carrier.

Compositions made by the process of the invention may be administered locally, topically or systemically, for example orally (enterally), by injection or infusion, intravenously or intraarterially (including by intravascular or other perivascular devices/dosage forms (e.g. stents)), intramuscularly, intraosseously, intracerebrally, intracerebroventricularly, intrasynovially, intrasternally, intrathecally, intralesionally, intracranially, intratumorally, cutaneously, intracutaneous, subcutaneously, transmucosally (e.g. sublingually or buccally), rectally, transdermally, nasally, pulmonarily (e.g. by inhalation, tracheally or bronchially), topically, or by any other parenteral route, such as subcutaneously or intramuscularly, optionally in the form of a pharmaceutical (or veterinary) preparation comprising the compound in a pharmaceutically (or veterinarily) acceptable dosage form.

The incorporation of compositions made by the process of the invention into pharmaceutical formulations may be achieved with due regard to the intended route of administration and standard pharmaceutical practice. Pharmaceutically acceptable excipients, such as carriers may be chemically inert to the biologically-active agent and may have no detrimental side effects or toxicity under the conditions of use. Such pharmaceutically acceptable carriers may also impart an immediate, or a modified, release of compositions made by the process of the invention.

Pharmaceutical (or veterinary) formulations comprising compositions made by the process of the invention may include particles of different types, for example particles comprising different active ingredients, comprising different functionalization (as described hereinbefore), particles of different sizes, and/or different thicknesses of the layers of coating materials, or a combination thereof. By combining, in a single pharmaceutical formulation, particles with different coating thicknesses and/or different core sizes, the drug release following administration to patient may be controlled (e.g. varied or extended) over a specific time period.

For peroral administration (i.e. administration to the gastrointestinal tract by mouth with swallowing), compositions made by the process of the invention may be formulated in a variety of dosage forms. Pharmaceutically acceptable carriers or diluents may be solid or liquid. Solid preparations include granules (in which granules may comprise some or all of the plurality of particles of a composition of the invention in the presence of e.g. a carrier and other excipients, such as a binder or pH adjusting agents), compressed tablets, pills, lozenges, capsules, cachets, etc. Carriers include materials that are well known to those skilled in the art, including those disclosed hereinbefore in relation to the formulation of biologically active agents within cores, as well as magnesium carbonate, pectin, dextrin, starch, gelatin, tragacanth, methylcellulose, sodium carboxymethylcellulose, a low melting wax, cocoa butter, lactose, microcrystalline cellulose, low-crystalline cellulose, and the like.

Solid dosage forms may comprise further excipients, such as flavouring agents, lubricants, binders, preservatives, disintegrants, and/or encapsulating materials. For example, compositions made by the process of the invention may be encapsulated e.g. in a soft or hard shell capsule, e.g. a gelatin capsule.

Compositions made by the process of the invention formulated for rectal administration may include suppositories that may contain, for example, a suitable non-irritating excipient, such as cocoa butter, synthetic glyceride esters or polyethylene glycols, which are solid at ordinary temperatures, but which liquefy and/or dissolve in the rectal cavity to release the particles of the compositions made by the process of the invention.

For parenteral administration, such as subcutaneous and/or intramuscular injections, the compositions made by the process of the invention may be in the form of sterile injectable and/or infusible dosage forms, for example, sterile aqueous or oleaginous suspensions of compositions made by the process of the invention.

Such suspensions may be formulated in accordance with techniques that are well known to those skilled in the art, by employing suitable dispersing or wetting agents (e.g. Tweens, such as Tween 80), and suspending agents.

Non-toxic parenterally-acceptable diluents also include solutions of 1,3-butanediol, mannitol, Ringer's solution, isotonic sodium chloride solution, sterile, fixed oils (including any bland fixed oil, such as synthetic mono- or diglycerides). Fatty acids, such as oleic acid and its glyceride derivatives may be used in the preparation of injectable formulations, as well as natural pharmaceutically-acceptable oils, such as olive oil or castor oil, and their polyoxyethylated versions, and pH adjusting agents. These oil suspensions may also contain a long-chain alcohol diluent or dispersant.

Compositions made by the process of the invention suitable for injection may also comprise compositions in the form of a liquid, a sol or a gel (e.g. comprising hyaluronic acid), which is administrable via a surgical administration apparatus, e.g. a needle, a catheter or the like, to form a depot formulation. The use of compositions made by the process of the invention may control the dissolution rate and the pharmacokinetic profile by reducing any burst effect as hereinbefore defined and/or by reducing the Cmax in a plasma concentration-time profile, and thus increasing the length of release of biologically active ingredient from that formulation.

Compositions made by the process of the invention may be contained within a reservoir and an injection or infusion means, wherein coated particles and carrier systems are housed separately and in which admixing occurs prior to and/or during injection or infusion.

Compositions made by the process of the invention may also be formulated for inhalation, e.g. as an inhalation powder for use with a dry powder inhaler (see, for example, those described by Kumaresan et al, Pharma Times, 44, 14 (2012) and Mack et al., Inhalation, 6, 16 (2012)), the relevant disclosures thereof are hereby incorporated by reference. Suitable particle sizes for the plurality of particles in a composition of the invention for use in inhalation to the lung are in the range of about 2 to about 10 μm.

Compositions made by the process of the invention may also be formulated for administration topically to the skin, or to a mucous membrane. For topical application, the pharmaceutical formulations may be provided in the form of e.g. a lotion, a gel, a paste, a tincture, a transdermal patch, a gel for transmucosal delivery, all of which may comprise a composition of the invention. The composition may also be formulated with a suitable ointment containing a composition of the invention suspended in a carrier, such as a mineral oil, liquid petroleum, white petroleum, propylene glycol, polyoxyethylene polyoxypropylene compound, emulsifying wax or water. Suitable carrier for lotions or creams include mineral oils, sorbitan monostearate, polysorbate 60, cetyl esters wax, cetaryl alcohol, 2-octyldodecanol, benzyl alcohol and water.

Pharmaceutical formulations may comprise between about 1% to about 99%, such as between about 10% (such as about 20%, e.g. about 50%) to about 90% by weight of the composition of the invention, with the remainder made up by pharmaceutically acceptable excipients.

In any event, compositions made by the process of the invention, may be formulated with conventional pharmaceutical additives and/or excipients used in the art for the preparation of pharmaceutical formulations, and thereafter incorporated into various kinds of pharmaceutical preparations and/or dosage forms using standard techniques (see, for example, Lachman et al, ‘The Theory and Practice of Industrial Pharmacy’, Lea & Febiger, 3^{rd} edition (1986); ‘Remington: The Science and Practice of Pharmacy’, Troy (ed.), University of the Sciences in Philadelphia, 21^{st} edition (2006); and/or ‘Aulton's Pharmaceutics: The Design and Manufacture of Medicines’, Aulton and Taylor (eds.), Elsevier, 4th edition, 2013), and the documents referred to therein, the relevant disclosures in all of which documents are hereby incorporated by reference. Otherwise, the preparation of suitable formulations may be achieved non-inventively by the skilled person using routine techniques.

According to a further aspect of the invention there is provided a process for the preparation of a pharmaceutical or veterinary formulation which comprises mixing together coated particles prepared as described herein with a pharmaceutically-acceptable or a veterinarily-acceptable adjuvant, diluent or carrier.

It is preferred that such formulations are injectable and/or infusible and therefore comprise one or more compositions made by the process of the invention suspended in a pharmaceutically-acceptable or a veterinarily-acceptable aqueous and/or oleaginous carrier.

There is further provided an injectable and/or infusible dosage form comprising a compositions made by the process of the invention contained within a reservoir and an injection or infusion means. In this respect, compositions made by the process of the invention can be stored prior to being loaded into a suitable injectable and/or infusible dosing means (e.g. a syringe with a needle for injection) or may be prepared immediately prior to loading into such a dosing means.

There is thus further provided a kit of parts comprising:
(a) a composition made by the process of the invention; and
(b) a pharmaceutically-acceptable or a veterinarily-acceptable carrier system,

as well as a kit of parts comprising a composition made by the process of the invention along with instructions to the end user to admix those particles with pharmaceutically-acceptable or a veterinarily-acceptable aqueous and/or oleaginous carrier system.

There is further provided a pre-loaded injectable and/or infusible dosage form as described in above, but modified by comprising at least two chambers, within one of which chamber is located composition made by the process of the invention and within the other of which is located a pharmaceutically-acceptable or a veterinarily-acceptable carrier system, wherein admixing, giving rise to a suspension or otherwise, occurs prior to and/or during injection or infusion.

Wherever the word ‘about’ is employed herein, for example in the context of amounts (e.g. concentrations, dimensions (sizes and/or weights), size ratios, aspect ratios, proportions or fractions), temperatures or pressures, it will be appreciated that such variables are approximate and as such may vary by ±15%, such as ±10%, for example ±5% and preferably ±2% (e.g. ±1%) from the numbers specified herein. This is the case even if such numbers are presented as percentages in the first place (for example ‘about 15%’ may mean±15% about the number 10, which is anything between 8.5% and 11.5%).

Compositions made by the process of the invention allow for the formulation of a large diversity of pharmaceutically active compounds. Compositions made by the process of the invention may be used to treat effectively a wide variety of disorders depending on the biologically active agent that is included.

Compositions made by the process of the invention may further be formulated in the form of injectable suspension of coated particles with a size distribution that is both even and capable of forming a stable suspension within the injection liquid (i.e. without settling) and may be injected through a needle.

Furthermore, compositions made by the process of the invention can be stored under normal storage conditions, and maintain their physical and/or chemical integrity.

The phrase ‘maintaining physical and chemical integrity’ essentially means chemical stability and physical stability.

By ‘chemical stability’, we include that any compositions made by the process of the invention may be stored (with or without appropriate pharmaceutical packaging), under normal storage conditions, with an insignificant degree of chemical degradation or decomposition.

By ‘physical stability’, we include that the any compositions made by the process of the invention may be stored (with or without appropriate pharmaceutical packaging), under normal storage conditions, with an insignificant degree of physical transformation, such as sedimentation as described above, or changes in the nature and/or integrity of the coated particles, for example in the coating itself or the active ingredient (including dissolution, solvatisation, solid state phase transition, etc.).

Examples of ‘normal storage conditions’ for compositions made by the process of the invention include temperatures of between about −50° C. and about +80° C. (preferably between about −25° C. and about +75° C., such as about 50° C.), and/or pressures of between about 0.1 and about 2 bars (preferably atmospheric pressure), and/or exposure to about 460 lux of UV/visible light, and/or relative humidities of between about 5 and about 95% (preferably about 10 to about 40%), for prolonged periods (i.e. greater than or equal to about twelve, such as about six months).

Under such conditions, compositions made by the process of the invention may be found to be less than about 15%, more preferably less than about 10%, and especially less than about 5%, chemically and/or physically degraded/decomposed, as appropriate. The skilled person will appreciate that the above-mentioned upper and lower limits for temperature and pressure represent extremes of normal storage conditions, and that certain combinations of these extremes will not be experienced during normal storage (e.g. a temperature of 50° C. and a pressure of 0.1 bar).

Furthermore, compositions made by the process of the invention may provide a release and/or pharmacokinetic profile that minimizes any burst effect and/or minimize Cmax, which is characterised by a concentration maximum shortly after administration.

The compositions and processes described herein may have the advantage that, in the treatment of a relevant condition with a particular biologically active agent, they may be more convenient for the physician and/or patient than, be more efficacious than, be less toxic than, have a broader range of activity than, be more potent than, produce fewer side effects than, or that it may have other useful pharmacological properties over, any similar treatments that may be described in the prior art for the same active ingredient.

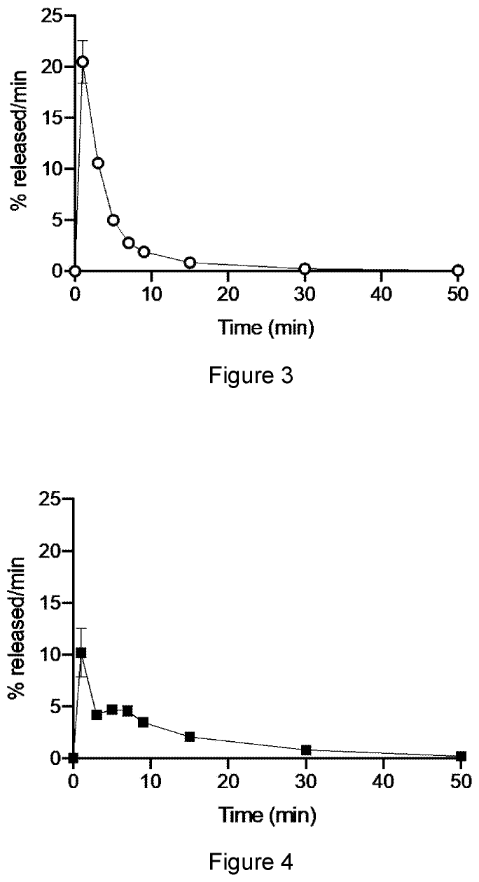

The invention is illustrated, but in no way limited, by the following examples with reference to the attached figures, in which FIGS. 1 and 2 are TEM images that show clearly visible physical interfaces (regions of higher electron permeability) that are formed by employing the process that is described herein; and FIGS. 3 and 4 show drug release profiles against time for samples obtained according to the examples.

### EXAMPLES

### Comparative Example 1

Coated Azacitidine Microparticles I

Microparticles of azacitidine (Olon SpA, Rodano, Italy) were prepared by jet-milling (by Catalent, in Malvern, Pa. (USA)). The mean diameter of the jet-milled azacitidine particles was 1.2 μm as determined by laser diffraction (Sympatec, Helos (H1672) and Rodos, R3, Clausthal-Zellerfeld, Germany).

The powder was loaded to an ALD reactor (Picosun, SUNALE™ R-series, Espoo, Finland). 35 ALD cycles were performed at a reactor temperature of 50° C. Diethyl zinc and water were used as precursors, forming a first layer of zinc oxide. The first layer was about 5 nm in thickness (as estimated from the number of ALD cycles).

The powder was removed from the reactor and deagglomerated by means of forcing the powder through a metal sieve with a 20 μm mesh size using a rubber spatula.

The resultant deagglomerated powder was re-loaded into the ALD reactor and further 35 ALD cycles were performed as before forming a second layer of zinc oxide, extracted from the reactor and deagglomerated by means of manual sieving as above, reloaded to form a third layer, deagglomerated and the reloaded to a final, fourth layer.

To determine the drug load (i.e. w/w % of azacitidine in the powder), HPLC (Prominence-i (Shimadzu, Japan) equipped with a diode array detector (Shimadzu, Japan) set at 210 nm was employed using a 4.6×250 mm, 3 μm particles, C18 column (Luna, Phenomenex, USA)). The nanoshell coatings were dissolved in 1 M phosphoric acid and the slurry was diluted to dissolve the azacitidine by dilution with 1 g/L of sodium bisulfite in water, before filtration (0.2 μm RC, Lab Logistics Group, Germany) and further analyzed with HPLC (n=2). The drug load was determined as 64.7%.

### Example 1

Coated Azacitidine Microparticles II

Corresponding coated microparticles of azacitidine were prepared as described in Comparative Example 1 above with the exception that the powder was sourced from MSN Labs (India), the particles had a mean diameter of 5.5 μm (as determined by laser diffraction (Shimadzu, SALD-7500nano, Kyoto, Japan), and deagglomeration was carried out by sieving through a nylon sieve with a mesh size of 20 μm using a sonic sifter (Tsutsui Scientific Instruments Co., Ltd., SW-20AT, Tokyo, Japan) to shake the powder through the sieve. The drug load was determined as 74.5%

### Example 2

In Vitro Drug Release

In vitro release studies for the particles of Comparative Example 1 and Example 1 were conducted using a Sotax CE 7smart USP 4 apparatus (Sotax AG, Switzerland) linked to a CP 7-35 piston pump (Sotax AG, Switzerland) and a C613 fraction collector (Sotax AG, Switzerland).

Flow-through cells with a 22.6 mm diameter were prepared with a 5 mm ruby bead in the tip of the cell cone, in which the suspended samples were introduced.

The samples were analyzed in duplicates with a sample amount corresponding to 50 mg azacitidine per cell. The samples (33.3 mg azacitidine/mL) were dispersed by vortexing in 0.1% Tween 20+0.25% Na-CMC in saline (0.9% NaCl) phosphate buffer with a pH of 7.2.

The apparatus was used in an open-loop set-up, in which fresh 20 mM PIPES, pH 7.2 dissolution medium was continuously introduced into the system. The temperature of the water bath was set at 37° C.±0.5° C. and the flow rate of media was set at 16 mL/min. The medium was filtered before leaving the flow through cells using two Whatman glass microfiber filters, GF/F and GF/D (d=25 mm, Sigma-Aldrich/Merck KGaA, Germany). The collected fractions of the release medium were analyzed for azacitidine content using HPLC, using the same setup as was used for the drug load analysis described above.

FIGS. 3 and 4 show the respective azacitidine release profiles (percentage of azacitidine released per minute versus sampling time in the Sotax apparatus for samples obtained by Comparative Example 1, and Example 1, respectively.

It can be seen that Comparative Example 1 has a higher initial (burst) release than Example 1.

1. A process for the preparation of composition in the form of a plurality of particles having a weight-, number-, and/or volume-based mean diameter that is between amount 10 nm and about 700 μm, which particles comprise:
(a) solid cores; and
(b) two or more sequentially applied, discrete layers, each of which comprises at least one separate coating material, and which two or more layers together surround, enclose and/or encapsulate said cores,
which process comprises the sequential steps of:
(1) applying an initial layer of at least one coating material to said solid cores by way of a gas phase deposition technique;
(2) discharging the coated particles from the gas phase deposition reactor and subjecting the coated particles to agitation to disaggregate particle aggregates formed during step (1) by way of mechanical sieving technique;
(3) reintroducing the disaggregated, coated particles from step (2) into the gas phase deposition reactor and applying a further layer of at least one coating material to the reintroduced particles; and
(4) optionally repeating steps (2) and (3) one or more times to increase the total thickness of the at least one coating material that enclose(s) said solid core.

2. The process as claimed in claim 1, wherein the cores comprise a biologically active agent and/or a pharmaceutically-acceptable excipient.

3. The process as claimed in claim 2, wherein the carrier/excipient material is a sugar or a sugar alcohol and/or is a pH modifying agent.

4. The process as claimed in claim 1, wherein the cores consist essentially of biologically active agent.

5. The process as claimed in claim 1, wherein the biologically active agent is selected from an analgesic, an anaesthetic, an anti-ADHD agent, an anorectic agent, an antiaddictive agent, an antibacterial agent, an antimicrobial agent, an antifungal agent, an antiviral agent, an antiparasitic agent, an antiprotozoal agent, an anthelmintic, an ectoparasiticide, a vaccine, an anticancer agent, an antimetabolite, an alkylating agent, an antineoplastic agent, a topoisomerase, an immunomodulator, an immunostimulant, an immunosuppressant, an anabolic steroid, an anticoagulant agent, an antiplatelet agent, an anticonvulsant agent, an antidementia agent, an antidepressant agent, an antidote, an antihyperlipidemic agent, an antigout agent, an antimalarial, an antimigraine agent, an anti-inflammatory agent, an antiparkinson agent, an antipruritic agent, an antipsoriatic agent, an antiemetic, an anti-obesity agent, an anthelmintic, an antiasthma agent, an antibiotic, an antidiabetic agent, an antiepileptic, an antifibrinolytic agent, an antihemorrhagic agent, an antihistamine, an antitussive, an antihypertensive agent, an antimuscarinic agent, an antimycobacterial agent, an antioxidant agent, an antipsychotic agent, an antipyretic, an antirheumatic agent, an antiarrhythmic agent, an anxiolytic agent, an aphrodisiac, a cardiac glycoside, a cardiac stimulant, an entheogen, an entactogen, an euphoriant, an orexigenic, an antithyroid agent, an anxiolytic sedative, a hypnotic, a neuroleptic, an astringent, a bacteriostatic agent, a beta blocker, a calcium channel blocker, an ACE inhibitor, an angiotensin II receptor antagonist, a renin inhibitor, a beta-adrenoceptor blocking agent, a blood product, a blood substitute, a bronchodilator, a cardiac inotropic agent, a chemotherapeutic, a coagulant, a corticosteroid, a cough suppressant, a diuretic, a deliriant, an expectorant, a fertility agent, a sex hormone, a mood stabilizer, a mucolytic, a neuroprotective, a nootropic, a neurotoxin, a dopaminergic, an antiparkinsonian agent, a free radical scavenging agent, a growth factor, a fibrate, a bile acid sequestrants, a cicatrizant, a glucocorticoid, a mineralcorticoid, a haemostatic, a hallucinogen, a hypothalamic-pituitary hormone, an immunological agent, a laxative agent, a antidiarrhoeals agent, a lipid regulating agent, a muscle relaxant, a parasympathomimetic, a parathyroid calcitonin, a serenic, a statin, a stimulant, a wakefulness-promoting agent, a decongestant, a dietary mineral, a biphosphonate, a cough medicine, an ophthamological, an ontological, a H1 antagonist, a H2 antagonist, a proton pump inhibitor, a prostaglandin, a radio-pharmaceutical, a hormone, a sedative, an anti-allergic agent, an appetite stimulant, a steroid, a sympathomimetic, a thrombolytic, a thyroid agent, a vasodilator, a xanthine, an erectile dysfunction improvement agent, a gastrointestinal agent, a histamine receptor antagonist, a keratolytic, an antianginal agent, a non-steroidal antiinflammatory agent, a COX-2 inhibitor, a leukotriene inhibitor, a macrolide, a NSAID, a nutritional agent, an opioid analgesic, an opioid antagonist, a potassium channel activator, a protease inhibitor, an antiosteoporosis agent, a cognition enhancer, an antiurinary incontinence agent, a nutritional oil, an antibenign prostate hypertrophy agent, an essential fatty acid, a non-essential fatty acid, a cytokine, a peptidomimetic, a peptide, a protein, a radiopharmaceutical, a senotherapeutic, a toxoid, a serum, an antibody, a nucleoside, a nucleotide, a vitamin, a portion of genetic material, a nucleic acid, or a mixture of any of these.

6. The process as claimed in claim 1, wherein the weight-, number-, or volume-, based mean diameter of the cores is between amount 1 μm and about 50 μm.

7. The process as claimed in claim 1, wherein between 3 and 10 discrete layers of coating material are applied to the core sequentially.

8. The process as claimed in claim 1, wherein, the total thickness of the discrete layers of coating material is between about 0.5 nm and about 2 μm.

9. The process as claimed in claim 1, wherein the maximum thickness of an individual discrete layer of coating material is about 1 hundredth of the weight-, number-, or volume-based mean diameter of the core, including any other previously-applied discrete layers of coating material that are located between said individual discrete layer and the outer surface of the core.

10. The process as claimed in claim 1, wherein the coating materials of the one or more discrete layers comprise one or more inorganic coating materials.

11. The process as claimed in claim 10, wherein the coating materials comprise one or more metal-containing, or metalloid-containing, compounds.

12. The process as claimed in claim 10, wherein the compounds comprise a hydroxide and/or an oxide.

13. The process as claimed in claim 10, wherein the one or more coating materials comprise aluminium oxide, titanium dioxide, zinc sulphide and/or zinc oxide.

14. The process as claimed in claim 10, wherein the one or more coating material comprise zinc oxide.

15. The process as claimed in claim 1, which comprises applying the separate layers of coating materials to cores, and/or previously-coated cores, by atomic layer deposition.

16. The process as claimed in claim 15, wherein the mechanical sieving comprises vibration or shaking of the sieve.

17. The process as claimed in claim 15, wherein the mechanical sieving comprises sonic sifting.

18. The process as claimed in claim 1, which process comprises a further step of resuspending separated particles in a solvent, with or without the presence of one or more pharmaceutically acceptable excipients.

19. The process as claimed in claim 2, wherein the biologically-active agent is an anti-cancer agent.

20. The process as claimed in claim 2, wherein the biologically-active agent is azacitidine.

21. A composition obtainable by way of a process as defined in claim 1.

22. (canceled)

23. A pharmaceutical or veterinary formulation comprising a composition as defined in claim 21 and a pharmaceutically-acceptable or a veterinarily-acceptable adjuvant, diluent or carrier.

24. The formulation as claimed claim 23 in the form of a sterile injectable and/or infusible dosage form.

25. The formulation as claimed or claim 24 in the form of a liquid, a sol or a gel, administrable via a surgical administration apparatus that forms a depot formulation.

26. A process as for the preparation of a formulation as defined in claim 23, which comprises admixing the composition as defined in with the relevant pharmaceutically-acceptable or veterinarily-acceptable adjuvant, diluent or carrier.

27-28. (canceled)

29. A method of treatment of cancer, which method comprises administration of a composition as claimed in claim 21, in which the biologically active agent is an anti-cancer agent, to patient in need of such treatment.

30. (canceled)

31. A method of treatment of cancer, which method comprises administration of a formulation as claimed in claim 23, in which the biologically active agent is an anti-cancer agent, to patient in need of such treatment.
